(19) United States
(12) Patent Application Publication
(10) Pub. No.: US 2016/0010086 A1
(43) Pub. Date: Jan. 14, 2016

(54) OLIGOMERIC COMPOUNDS AND EXCIPIENTS

(71) Applicant: ISIS PHARMACEUTICALS, INC., CARLSBAD, CA (US)

(72) Inventors: C. FRANK BENNETT, CARLSBAD, CA (US); RICHARD S. GEARY, CARLSBAD, CA (US); ERIC E. SWAYZE, ENCINITAS, CA (US); ANDREW M. SIWKOWSKI, CARLSBAD, CA (US)

(73) Assignee: ISIS PHARMACEUTICALS, INC., CARLSABAD, CA (US)

(21) Appl. No.: 14/558,621

(22) Filed: Dec. 2, 2014

Related U.S. Application Data
(62) Division of application No. 13/148,291, filed on Jun. 29, 2012, now abandoned.
(60) Provisional application No. 61/150,708, filed on Feb. 6, 2009.

(51) Int. Cl. C12N 15/113; A61K 31/721; A61K 47/36

(52) U.S. Cl. CPC C12N 15/113; A61K 47/36; A61K 31/721; C12N 2310/11; C12N 2320/30

(57) ABSTRACT

The present invention provides method of optimizing the efficacy and potency of antisense compounds. In certain embodiments, the invention provides assays useful for determining favorable oligonucleotide characteristics and excipients for improved cellular uptake.

### CROSS REFERENCED TO RELATED APPLICATIONS

This application is a divisional of U.S. application Ser. No. 13/148,291, filed Jun. 29, 2014, which is a U.S. National Phase filing under 35 U.S.C. 371 claiming priority to International Application No. PCT/US2010/023383, filed Feb. 5, 2010; which claims priority under 35 U.S.C. 119(e) to U.S. Provisional 61/150,708, filed Feb. 6, 2009; each of which is incorporated herein by reference in its entirety.

### FIELD OF THE INVENTION

The present invention provides compounds, systems, and methods for increasing productive uptake of antisense compounds in cells.

### SEQUENCE LISTING

The present application is being filed along with a Sequence Listing in electronic format. The Sequence Listing is provided as a file entitled CORE0081USD1SEQ_ST25.txt, created on Dec. 2, 2014 which is 4 Kb in size. The information in the electronic format of the sequence listing is incorporated herein by reference in its entirety.

### BACKGROUND OF THE INVENTION

Antisense compounds have been used to modulate target nucleic acids. Antisense compounds comprising a variety of modifications and motifs have been reported. In certain instances, such compounds are useful as research tools and as therapeutic agents. Certain DNA-like oligomeric compounds have been shown to reduce protein expression. Certain RNA-like compounds are known to inhibit protein expression in cells. Such RNA-like compounds function, at least in part, through the RNA-inducing silencing complex (RISC). RNA-like compounds may be single-stranded or double-stranded. Antisense compounds have also been shown to alter processing of pre-mRNA and to modulate non-coding RNA molecules. Compounds and methods that increase productive uptake of antisense compounds in cells are desired.

### SUMMARY OF THE INVENTION

The present invention provides compounds, systems, and methods for increasing productive uptake of antisense compounds in cells. For example, in some embodiments, the present invention provides excipients that increase the uptake and/or activity of antisense compounds in cells and tissues in vitro and in vivo. Thus, the present invention provides compounds, systems, and methods that facilitate improved use of antisense compounds for research and therapeutic applications.

In some embodiments, the present invention provides methods of treating a subject comprising: administering a pharmaceutical composition to a subject, wherein the pharmaceutical composition comprises, or consists essentially of, antisense oligonucleotides and an excipient (e.g, polyanionic polymer). In some embodiments, the excipient is present in the composition at a concentration between 0.0001 and 9.0 uM (e.g., about 0.001 mM . . . 0.01 mM . . . 0.1 mM . . . 1 mM . . . 10 mM . . . 50 mM . . . and 90 mM). In some embodiments, the excipient is present in the composition at a concentration between 0.0001 and 1000.0 mM (e.g., about 0.0001 mM 0.0001 mM . . . 0.01 mM . . . 0.1 mM . . . 1 mM . . . 10 mM . . . 50 mM . . . 100 mM . . . 150 mM . . . 200 mM . . . 500 mM . . . 750 mM . . . and 1000 mM). In certain embodiments, the present invention provides compositions comprising, or consisting essentially of, antisense oligonucleotides and an excipient (e.g., polyanionic polymer) for use as a medicament.

In some embodiments, the present invention provides methods of treating a subject comprising: administering a pharmaceutical composition to a subject, wherein the pharmaceutical composition comprises, or consists essentially of, antisense oligonucleotides and a branched polysaccharide or glucan derivative (e.g., branched glucan derivative, dextran derivative, destran ester, sulfur containing dextran derivative, sulfur containing dextran ester, sulfated dextran derivative, dextran sulfate, sulfated polyvinyl alcohol (PVAS), polyvinyl sulfate (PVS), PRO-2000, sulfated copolymers of acrylic acid and vinyl alcohol (PAVAS). In further embodiments, the present invention provides compositions comprising (or consists essentially of) a antisense oligonucleotides and a branched polysaccharide or glucan derivative (e.g., branched glucan derivative, dextran derivative, destran ester, sulfur containing dextran derivative, sulfur containing dextran ester, sulfated dextran derivative, dextran sulfate, sulfated polyvinyl alcohol (PVAS), polyvinyl sulfate (PVS), PRO-2000, sulfated copolymers of acrylic acid and vinyl alcohol (PAVAS) for use as a medicament.

In some embodiments, the present invention provides methods of treating a subject comprising: administering a pharmaceutical composition to a subject, wherein the pharmaceutical composition comprises, or consists essentially of, an antisense oligonucleotide and an excipient oligomeric compound. In certain embodiments, the excipient oligomeric compound comprises an excipient oligonucleotide. In further embodiments, the excipient oligonucleotide consists of 5-100 linked nucleosides or nucleoside analogues. In particular embodiments, the excipient oligonucleotide comprises at least one unmodified deoxyribonucleoside. In certain embodiments, the excipient oligonucleotide comprises at least one unmodified ribonucleoside. In further embodiments, the excipient oligonucleotide comprises at least one modified nucleoside or nucleoside analogue. In other embodiments, the excipient oligonucleotide comprises a plurality of modified nucleosides or nucleoside analogues. In particular embodiments, substantially all of the nucleoside or nucleoside analogues of the modified excipient oligonucleotide are modified nucleosides or nucleoside analogues. In some embodiments, all of the nucleoside or nucleoside analogues of the modified excipient oligonucleotide are modified nucleosides or nucleoside analogues. In particular embodiments, the excipient oligonucleotide comprises at least one modified linkage. In additional embodiments, each linkage of the excipient oligonucleotide is a modified linkage. In other embodiments, at least one modified linkage of the excipient oligonucleotide is a phosphorothioate linkage. In particular embodiments, the each modified linkage of the excipient oligonucleotide is a phosphorothioate linkage. In other embodiments, each modified linkage of the excipient oligonucleotide is a phosphorothioate linkage.

In some embodiments, the present invention provides methods of treating a subject comprising: administering a pharmaceutical composition to a subject, wherein the pharmaceutical composition comprises, or consists essentially of, antisense oligonucleotide and dextran sulfate or a dextran sulfate analogue or derivative. In further embodiments, the present invention provides compositions comprising, or consisting essentially of, a antisense oligonucleotides and dextran sulfate or a dextran sulfate analogue or derivative for use as a medicament.

In some embodiments, the present invention provides methods of treating a subject comprising: administering a pharmaceutical composition to a subject, wherein the pharmaceutical composition comprises, or consists essentially of, an antisense oligonucleotide and an excipient (e.g, polyanionic polymer), wherein the composition is block co-polymer free. In some embodiments, the present invention provides compositions comprising antisense oligonucleotides and an excipient (e.g, polyanionic polymer) for use as a medicament, wherein the composition is block co-polymer free.

In some embodiments, the present invention provides methods of treating a subject comprising: administering a pharmaceutical composition to a subject, wherein the pharmaceutical composition consists essentially of antisense an oligonucleotide and an excipient (e.g., polyanionic polymer). In further embodiments, such compositions further consist essentially of purified water or saline solution. In additional embodiments, the present invention provides compositions comprising, or consisting essentially of, antisense oligonucleotides and an excipient (e.g., polyanionic polymer) for use as a medicament, which may further comprise purified water or saline solution.

In other embodiments, the present invention provides methods of treating a subject comprising: administering a pharmaceutical composition to a subject, wherein the composition comprises an antisense oligonucleotide and an excipient (e.g., polyanionic polymer), wherein the excipient has a molecular weight of 4,000-50,000 daltons (4,000 . . . 10,000 . . . 20,000 . . . 25,000 . . . 30,000 . . . 40,000 . . . and 50,000). In further embodiments, the present invention provides compositions comprising antisense oligonucleotides and an excipient (e.g., polyanionic polymer) for use as a medicament, wherein the excipient has a molecular weight of 4,000-30,000 daltons (4,000 . . . 7,000 . . . 10,000 . . . 13,000 . . . 16,000 . . . 20,000 . . . and 30,000).

In some embodiments, the present invention provides methods of treating a subject comprising: administering a pharmaceutical composition to a subject, wherein the composition comprises, consists essentially of, or consists of: i) an antisense oligonucleotides, ii) an excipient (e.g., a polyanionic polymer), and iii) purified water or saline solution.

In certain embodiments, the present invention provides methods of treating a subject comprising: administering a pharmaceutical composition to a subject, wherein the pharmaceutical composition comprises an antisense oligonucleotides and an excipient (e.g., polyanionic polymer), wherein the antisense oligonucleotide is present in the composition at a first concentration and the excipient is present in the composition at a second concentration, and wherein the ratio of the first concentration to the second concentration is between 1000:1 and 1:1000 (e.g., 1000:1 . . . 700:1 . . . 500:1 . . . 200:1 . . . 100:1 . . . 75:1 . . . 50:1 . . . 25:1 . . . 10:1 . . . 5:1 . . . 1:1 . . . 1:5 . . . 1:10 . . . 1:25 . . . 1:50 . . . 1:75 . . . 1:100 . . . 1:200 . . . 1:500 . . . 1:700 . . . 1:900 . . . 1:1000). In further embodiments, the present invention provides compositions comprising an antisense oligonucleotide and an excipient (e.g., polyanionic polymer) for use as a medicament, wherein the antisense oligonucleotide is present in the composition at a first concentration and the excipient is present in the composition at a second concentration, and wherein the ratio of the first concentration to the second concentration is between 1000:1 and 1:1000 (e.g., 1000:1 . . . 700:1 . . . 500:1 . . . 200:1 . . . 100:1 . . . 75:1 . . . 50:1 . . . 25:1 . . . 10:1 . . . 5:1 . . . 1:1 . . . 1:5 . . . 1:10 . . . 1:25 . . . 1:50 . . . 1:75 . . . 1:100 . . . 1:200 . . . 1:500 . . . 1:700 . . . 1:900 . . . 1:1000).

In certain embodiments, the present invention provides methods of treating a subject comprising: a) administering a first composition to a subject, wherein the first composition comprises an excipient (e.g., polyanionic polymers), and b) after step a), administering a second composition to the subject, wherein the second composition comprises an antisense oligonucleotide. In particular embodiments, the first composition comprising an excipient is administered for a duration and/or dosage such that the effect of later administering the antisense oligonucleotide is increased (e.g., 1.5×, 2×, 3×, or more increased as compared to administering the antisense oligonucleotide alone). In certain embodiments, step a) is performed 5 minutes to 48 hours prior to step b) (e.g., 5 minutes . . . 30 minutes . . . 1 hour . . . 6 hours . . . 12 hours . . . 24 hours . . . 48 hours).

In other embodiments, the present invention provides pharmaceutical compositions made by an antisense oligonucleotide manufacturing method, such as those described herein.

In some embodiments, the present invention provides compositions comprising (or consisting of or consisting essentially of): a) an antisense oligonucleotide, b) an excipient (e.g., polyanionic polymer), and c) purified water or saline solution.

In some embodiments, the present invention provides systems comprising: a) a pharmaceutical composition comprising, or consisting essentially of, i) antisense oligonucleotides and a branched polysaccharide or glucan derivative (e.g., branched glucan derivative, dextran derivative, destran ester, sulfur containing dextran derivative, sulfur containing dextran ester, sulfated dextran derivative, dextran sulfate, sulfated polyvinyl alcohol (PVAS), polyvinyl sulfate (PVS), PRO-2000, sulfated copolymers of acrylic acid and vinyl alcohol (PAVAS), ii) a polyanionic polymer; and b) liquid container (e.g., a syringe vial or syringe), wherein the pharmaceutical composition is located within the liquid container.

In further embodiments, the present invention provides systems comprising: a) a pharmaceutical composition comprising: i) antisense oligonucleotides, and ii) dextran sulfate or a dextran sulfate analogue or derivative, and b) liquid container (e.g., a syringe vial or syringe), wherein the pharmaceutical composition is located within the liquid container.

In particular embodiments, the present invention provides systems comprising: a) a pharmaceutical composition comprising: i) antisense oligonucleotides, and ii) a polyanionic polymer, wherein the composition is block co-polymer free; and b) liquid container (e.g., a syringe vial or syringe), wherein the pharmaceutical composition is located within the liquid container.

In certain embodiments, the antisense oligonucleotides include one or more of: micro-RNA, antisense oligonucleotides, siRNA, catalytic oligonucleotides, and expressible gene sequences). In certain embodiments, the pharmaceutical compositions are block co-polymer free. In other embodiments, the pharmaceutical compositions further comprise purified water or saline solution. In particular embodiments, the subject is suffering from a disease, and wherein the administering is under conditions such that at least one symptom of the disease is reduced or eliminated. In other embodiments, the subject is a human. In certain embodiments, the subject is an animal (e.g., cow, pig, dog, cat, goat, horse, chicken, or other livestock). In certain embodiments, the polyanionic polymer has a molecular weight between 4,000-50,000 daltons (e.g., 5000 . . . 10000 . . . 20000 . . . 30000 . . . and 50000 daltons). In certain embodiments, the excipient is selected from the group consisting of: a branched glucan derivative, dextran derivative, destran ester, sulfur containing dextran derivative, sulfur containing dextran ester, sulfated dextran derivative, dextran sulfate, sulfated polyvinyl alcohol (PVAS), polyvinyl sulfate (PVS), PRO-2000, sulfated copolymers of acrylic acid and vinyl alcohol (PAVAS). In particular embodiments, the polyanionic polymer is not a nucleic acid (e.g., is a non-nucleic acid polyanionic polymer). In certain embodiments, the therapeutic effect (e.g., as measured by a decrease in target nucleic acid mRNA levels, protein levels, or protein activity) of said pharmaceutical composition is at least 1.125 greater (e.g., 1.125 . . . 1.5 . . . 2.0 . . . 4.0 . . . 10.0 . . . etc.) than administering the same composition without the excipient. In certain embodiments, the present invention provides formulations comprising an antisense oligomeric compound and an excipient (e.g., polyanionic polymer), wherein the excipient saturates a mechanism of unproductive accumulation in a cell.

In some embodiments, the present invention provides for the use of the pharmaceutical compositions of the present invention (e.g., containing antisense oligonucleotides and an excipient) for the manufacture of a medicament for the treatment of a certain disease or condition. In the present invention is not limited by the disease or condition. Examples of diseases or conditions include, but are not limited to: LDL-C reduction, coronary artery disease, diabetes, cancer, ulcerative colitis, multiple sclerosis, asthma, CMV retinitis, HCV infection, ocular disease, ALS, acromegaly, fibrosis, neurodegenerative diseases, arthritis, prostate cancer, feline viral outbreak, and HIV infection.

Exemplary antisense oligonucleotides include, but are not limited to: ISIS 3521 (Isis Pharmaceuticals), ISIS 2503 (Isis Pharmaceuticals), ISIS 5132 (Isis Pharmaceuticals), AP 12009 (Antisense Pharma), Oncomyc NG (AVI BioPharma), AVI 4557 (AVI BioPharma), Genasense (Genta), GEM 231 (Hybridon), GTI 2040 (Lorus Therapeutics), GTI 2501 (Lorus Therapeutics), LErafAON (NeoPharm), PAN 346 (Panacea Pharmaceuticals), HERZYME (Ribozyme Pharmaceuticals), ANGIOZYME (Ribozyme Pharmaceuticals), Resten NG (AVI BioPharma), E2F Decoy (Corgentech), ISIS 2302 (Isis Pharmaceuticals), ISIS 104838 (Isis Pharmaceuticals), AVI 4014 (AVI BioPharma), Durason (EpiGenesis), AVI 4126 (AVI BioPharma), ISIS 14803 (Isis Pharmaceuticals), HEPTAZYME (Ribozyme Pharmaceuticals), HepBzyme (Ribozyme Pharmaceuticals), PNAbiotics (Pantheco), NeuBiotics (AVI BioPharma), GEM 92 (Hybridon), and HGTV 43 (Enzo). In other embodiments, the oligonucleotide is an antisense oligonucleotide from Isis Pharamaceuticals, such as: MIPOMERSEN (ISIS 301012), ISIS 353512, BMS-PCSK9, ISIS 113715, ISIS 325568, ISIS 377131, ISIS388626, OGX-011, LY2181308, LY2275796, OGX-427, ALICAFORSEN (ISIS 2302), ATL/TV1102, AIR645 (ISIS 369645), Vitravene® (fomivirsen), iCo-007, ISIS 333611, ATL1103, and EXC001.

In certain embodiments, the antisense oligonucleotide consists of 10-30 linked nucleosides (e.g., 10 . . . 15 . . . 20 . . . 25 . . . and 30). In some embodiments, the antisense oligonucleotide is single-stranded. In other embodiments, the antisense oligonucleotide is double-stranded. In further embodiments, the antisense oligonucleotide comprises at least one modified nucleoside. In particular embodiments, the antisense oligonucleotide has at least one modified nucleoside is a 2′-modified nucleoside or a bicyclic nucleoside. In further embodiments, the at least one modified nucleoside is selected from among: 2′-MOE, 2′-F, 2′-OMe, and LNA. In additional embodiments, the antisense oligonucleotide is a gapmer. In further embodiments, the wing of the gapmer comprises linked 2′-MOE modified nucleosides and the gap of the gapmer comprises linked deoxyribonucleosides.

The present invention is not limited by the mode of administration of the pharmaceutical compositions to the subjection. In certain embodiment, the administering is by injection. In some embodiments, the injection is selected from intramuscular injection, subcutaneous injection, intravenous injection, intrathecal injection. In some embodiments, the administration is oral, topical, via inhalation, is aerosol, via enema, intravitreal, intrathecal, intravenous, subcutaneous, or topical.

### DESCRIPTION OF THE DRAWINGS

FIG. 1 provides a graph showing the effect of dextran sulfate on the activity of antisense molecules.

FIGS. 2a and 2b show the ability of antisense molecules with and without varying concentrations of dextran sulfate to inhibit SR-B1 mRNA levels in vivo.

### DETAILED DESCRIPTION OF THE INVENTION

It is to be understood that both the foregoing general description and the following detailed description are exemplary and explanatory only and are not restrictive of the present invention. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### I. DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, “nucleoside” refers to a glycosylamine comprising a heterocyclic base moiety and a sugar moiety. Nucleosides include, but are not limited to, naturally occurring nucleosides, abasic nucleosides, modified nucleosides, and nucleosides having mimetic bases and/or sugar groups. Nucleosides may be modified with any of a variety of substituents.

As used herein, “sugar moiety” means a natural or modified sugar ring or sugar surrogate.

As used herein, “nucleotide” refers to a nucleoside comprising a phosphate linking group. As used herein, nucleosides include nucleotides.

As used herein, “nucleobase” refers to the heterocyclic base portion of a nucleoside. Nucleobases may be naturally occurring or may be modified. In certain embodiments, a nucleobase may comprise any atom or group of atoms capable of hydrogen bonding to a base of another nucleic acid.

As used herein, “modified nucleoside” refers to a nucleoside comprising at least one modification compared to naturally occurring RNA or DNA nucleosides. Such modification may be at the sugar moiety and/or at the nucleobase.

As used herein, “bicyclic nucleoside” or “BNA” refers to a nucleoside wherein the sugar moiety of the nucleoside comprises a bridge connecting two carbon atoms of the sugar ring, thereby forming a bicyclic sugar moiety.

As used herein, “4′-2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, the term “deoxyribonucleotide” means a nucleotide having a hydrogen at the 2′ position of the sugar portion of the nucleotide. Deoxyribonucleotides may be modified with any of a variety of substituents.

As used herein, the term “ribonucleotide” means a nucleotide having a hydroxy at the 2′ position of the sugar portion of the nucleotide. Ribonucleotides may be modified with any of a variety of substituents.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein “oligonucleoside” refers to an oligonucleotide in which none of the internucleoside linkages contains a phosphorus atom. As used herein, oligonucleotides include oligonucleosides.

As used herein, “modified oligonucleotide” refers to an oligonucleotide comprising at least one modified nucleoside and/or at least one modified internucleoside linkage.

As used herein “internucleoside linkage” refers to a covalent linkage between adjacent nucleosides.

As used herein “naturally occurring internucleoside linkage” refers to a 3′ to 5′ phosphodiester linkage.

As used herein, “modified internucleoside linkage” refers to any internucleoside linkage other than a naturally occurring internucleoside linkage.

As used herein, “oligomeric compound” refers to a polymeric structure comprising two or more sub-structures. In certain embodiments, an oligomeric compound is an oligonucleotide. In certain embodiments, an oligomeric compound is a single-stranded oligonucleotide. In certain embodiments, an oligomeric compound is a double-stranded duplex comprising two oligonucleotides. In certain embodiments, an oligomeric compound is a single-stranded or double-stranded oligonucleotide comprising one or more conjugate groups and/or terminal groups.

As used herein, “duplex” refers to two separate oligomeric compounds that are hybridized together.

As used herein, “terminal group” refers to one or more atom attached to either, or both, the 3′ end or the 5′ end of an oligonucleotide. In certain embodiments a terminal group is a conjugate group. In certain embodiments, a terminal group comprises one or more additional nucleosides.

As used herein, “conjugate” refers to an atom or group of atoms bound to an oligonucleotide or oligomeric compound. In general, conjugate groups modify one or more properties of the compound to which they are attached, including, but not limited to pharmakodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleosides of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “antisense compound” refers to an oligomeric compound, at least a portion of which is at least partially complementary to a target nucleic acid to which it hybridizes. In certain embodiments, an antisense compound modulates (increases or decreases) expression or amount of a target nucleic acid. In certain embodiments, an antisense compound alters splicing of a target pre-mRNA resulting in a different splice variant. In certain embodiments, an antisense compound modulates expression of one or more different target proteins. Antisense mechanisms contemplated herein include, but are not limited to an RNase H mechanism, RNAi mechanisms, splicing modulation, translational arrest, altering RNA processing, inhibiting microRNA function, or mimicking microRNA function.

As used herein, “expression” refers to the process by which a gene ultimately results in a protein. Expression includes, but is not limited to, transcription, splicing, post-transcriptional modification, and translation.

As used herein, “RNAi” refers to a mechanism by which certain antisense compounds effect expression or amount of a target nucleic acid. RNAi mechanisms involve the RISC pathway.

As used herein, “RNAi compound” refers to an oligomeric compound that acts through an RNAi mechanism to modulate a target nucleic acid and/or protein encoded by a target nucleic acid. RNAi compounds include, but are not limited to double-stranded short interfering RNA (siRNA), single-stranded RNA (ssRNA), and microRNA, including microRNA mimics.

As used herein, “antisense oligonucleotide” refers to an antisense compound that is an oligonucleotide.

As used herein, “antisense activity” refers to any detectable and/or measurable activity attributable to the hybridization of an antisense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. For example, in certain embodiments, antisense activity is assessed by detecting and/or measuring the amount of target protein or the relative amounts of splice variants of a target protein. In certain embodiments, antisense activity is assessed by detecting and/or measuring the amount of target nucleic acids and/or cleaved target nucleic acids and/or alternatively spliced target nucleic acids. In certain embodiments, antisense activity is assessed by observing a phenotypic change in a cell or animal.

As used herein “detecting” or “measuring” in connection with an activity, response, or effect indicate that a test for detecting or measuring such activity, response, or effect is performed. Such detection and/or measuring may include values of zero. Thus, if a test for detection or measuring results in a finding of no activity (activity of zero), the step of detecting or measuring the activity has nevertheless been performed. For example, in certain embodiments, the present invention provides methods that comprise steps of detecting antisense activity, detecting toxicity, and/or measuring a marker of toxicity. Any such step may include values of zero.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is mRNA, pre-mRNA, non-coding RNA, pri-microRNA, pre-microRNA, mature microRNA, promoter-directed RNA, or natural antisense transcripts. For example, the target nucleic acid can be a cellular gene (or mRNA transcribed from the gene) whose expression is associated with a particular disorder or disease state, or a nucleic acid molecule from an infectious agent. In certain embodiments, target nucleic acid is a viral or bacterial nucleic acid.

As used herein, “target mRNA” refers to a pre-selected RNA molecule that encodes a protein.

As used herein, “target pre-mRNA” refers to a pre-selected RNA transcript that has not been fully processed into mRNA. Notably, pre-RNA includes one or more intron.

As used herein, “target microRNA” refers to a pre-selected non-coding RNA molecule about 18-30 nucleobases in length that modulates expression of one or more proteins or to a precursor of such a non-coding molecule.

As used herein, “target pdRNA” refers to refers to a pre-selected RNA molecule that interacts with one or more promoter to modulate transcription.

As used herein, “microRNA” refers to a naturally occurring, small, non-coding RNA that represses gene expression at the level of translation. In certain embodiments, a microRNA represses gene expression by binding to a target site within a 3′ untranslated region of a target nucleic acid. In certain embodiments, a microRNA has a nucleobase sequence as set forth in miRBase, a database of published microRNA sequences found at http://microrna.sanger.ac.uk/sequences/. In certain embodiments, a microRNA has a nucleobase sequence as set forth in miRBase version 10.1 released December 2007, which is herein incorporated by reference in its entirety. In certain embodiments, a microRNA has a nucleobase sequence as set forth in miRBase version 12.0 released September 2008, which is herein incorporated by reference in its entirety.

As used herein, “microRNA mimic” refers to an oligomeric compound having a sequence that is at least partially identical to that of a microRNA. In certain embodiments, a microRNA mimic comprises the microRNA seed region of a microRNA. In certain embodiments, a microRNA mimic modulates translation of more than one target nucleic acids.

As used herein, the term “uptake” or “taken up” refers to the ability of an oligomeric compound to enter a cell in a way that allows antisense activity.

As used herein, the term “accumulate” refers to the ability of an oligomeric compound to enter a cell, whether or not it is available for antisense activity. For example, if an oligomeric compound enters a cell, but is localized such that it is shielded from its target nucleic acid and no antisense activity is detected, the oligomeric compound has “accumulated” in the cell, but has not been “taken up.”

As used herein, the term “excipient” refers to a compound which, when present results in greater uptake of an antisense compound and/or greater antisense activity of an antisense compound.

As used herein, the term “polyanion” refers to a compound or chemical complex having at least two negative charges.

As used herein, the term “excipient oligomeric compound” refers to an oligomeric compound that has at least two negative charges and is not an antisense compound.

As used herein, the term “excipient oligonucleotide” refers to an oligonucleotide that has at least two negative charges and is not an antisense compound. In certain embodiments, an excipient oligonucleotide has a nucleobase sequence that is not complementary to any cellular nucleic acids. In certain embodiments, an excipient oligonucleotide comprises one or more abasic nucleosides. In certain embodiments, all of the nucleosides of an excipient oligonucleotide are abasic nucleosides.

As used herein, the term “motif” refers to a pattern of unmodified and modified nucleosides and/or linkages in an oligomeric compound. In certain embodiments, a motif can be described using a shorthand nomenclature comprising a series of numbers where each number represents the number of nucleosides of an oligomeric compound comprising a particular modification, where the first number represents the number of nucleosides of a type starting at the 5′ end of the oligomeric compound. For example: a 2-8-3 MOE-DNA gapmer is an oligonucleotide wherein the two 5′ terminal nucleosides are MOE-substituted nucleosides, the next eight nucleosides are unsubstituted DNA, and the three 3′ terminal nucleosides are MOE-substituted nucleosides. Linkage modifications can likewise be identified, for example the above 2-8-3 MOE gapmer could also have a 3-2-3-2-2 alternating phosphorothioate/phosphodiester, mixed backbone, wherein the first three linkages starting at the 5′ end (i.e., the linkages between the first and second nucleoside, the second and third nucleoside, and the third and fourth nucleoside) are each phosphorothioate, the next two are phosphodiester, the next three are phosphorothioate, the next two are phosphodiester, and the final two are phosphorothioate.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

As used herein, the term “expression” refers to all the functions and steps by which a gene's coded information is converted into structures present and operating in a cell. Such structures include, but are not limited to the products of transcription and translation.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric antisense compounds are gapmers having 2′-deoxynucleotides in the gap and nucleotides with high-affinity modifications in the wing.

As used herein, the term “cap structure” or “terminal cap moiety” refers to chemical modifications, which have been incorporated at either terminus of an antisense compound.

As used herein, “pharmaceutically acceptable salts” refers to salts of active compounds that retain the desired biological activity of the active compound and do not impart undesired toxicological effects thereto.

As used herein, “mitigation” refers to a lessening of at least one activity or one indicator of the severity of a condition or disease. The severity of indicators may be determined by subjective or objective measures which are known to those skilled in the art. In certain embodiments, the condition may be a toxic effect of a pharmaceutical agent.

As used herein, “pharmaceutical agent” refers to a substance that provides a effect when administered to a subject. In certain embodiments, a pharmaceutical agent provides a therapeutic benefit. In certain embodiments, a pharmaceutical agent provides a toxic effect.

As used herein, “therapeutic index” refers to the toxic dose of a drug for 50% of the population (TD₅₀) divided by the minimum effective dose for 50% of the population (ED₅₀). A high therapeutic index is preferable to a low one: this corresponds to a situation in which one would have to take a much higher amount of a drug to cause a toxic effect than the amount taken to cause a therapeutic benefit.

As used herein, “therapeutically effective amount” refers to an amount of a pharmaceutical agent that provides a therapeutic benefit to an animal.

As used herein, “administering” refers to providing a pharmaceutical agent to an animal, and includes, but is not limited to administering by a medical professional and self-administering.

As used herein, “co-administer” refers to administering more than one pharmaceutical agent to an animal. The more than one agent may be administered together or separately; at the same time or different times; through the same route of administration or through different routes of administration.

As used herein, “co-formulation” refers to a formulation comprising two or more distinct compounds. In certain embodiments, a co-formulation comprises two or more oligomeric compounds. In certain such embodiments, two or more oligomeric compound are oligomeric compounds of the present invention. In certain embodiments, one or more oligomeric compound present in a co-formulation is not a compound of the present invention. In certain embodiments, a co-formulation includes one or more non-oligomeric pharmaceutical agents. In certain embodiments, a co-formulation comprises one or more pharmaceutical agents and one or more excipients.

As used herein, “route of administration” refers to the means by which a pharmaceutical agent is administered to an animal.

As used herein, “pharmaceutical composition” refers to a mixture of substances suitable for administering to an animal. For example, a pharmaceutical composition may comprise an antisense oligonucleotide and a sterile aqueous solution.

As used herein, “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

As used herein, “animal” refers to a human or a non-human animal, including, but not limited to, mice, rats, rabbits, dogs, cats, pigs, and non-human primates, including, but not limited to, monkeys and chimpanzees.

As used herein, “parenteral administration,” refers to administration through injection or infusion. Parenteral administration includes, but is not limited to, subcutaneous administration, intravenous administration, or intramuscular administration.

As used herein, “subcutaneous administration” refers to administration just below the skin. “Intravenous administration” refers to administration into a vein.

As used herein, “active pharmaceutical ingredient” refers to the substance in a pharmaceutical composition that provides a desired effect.

As used herein, “prodrug” refers to a therapeutic agent that is prepared in an inactive form that is converted to an active form (i.e., drug) within the body or cells thereof by the action of endogenous enzymes or other chemicals and/or conditions.

As used herein, the term “excipient” refers to a compound that is co-administered with a pharmaceutical agent, that does not provide the biological effect of the therapeutic agent when administered in the absence of the therapeutic agent, but which modulates the amount or type of biological effect of a pharmaceutical agent.

As used herein, the term “glycan” refers to any macromolecule containing multiple saccharide units. A glycan most typically refers to a polysaccharide or oligosaccharide. A glycan may also typically refer to the carbohydrate portion of a glycoconjugate, such as a glycoprotein, glycolipid, or a proteoglycan. Glycans may be long (e.g. 40-4000 monosaccharide units) or short (e.g. 2-39 monosaccharide units), linear or branched, and made up or a single type of monosaccharide (e.g. cellulose is comprised of only glucose monomers) or multiple monosaccharides (e.g. heparin is comprised of two different repeating monosaccharides).

As used herein, the term “polysaccharide” refers to a polymer compound in which monosaccharides joined together by glycosidic bonds. The term “polysaccharide” typically refers to polymers of 11 or more monosaccharides, although shorter lengths are also included. Polymers of monosaccharides that are shorter in length than polysaccharides are typically referred to as oligosaccharides.

As used herein, the term “glycosaminoglycan” or “mucopolysaccharides” refers to any of a group of polysaccharides derived from an amino hexose. Gluycosaminoglycans are typically long unbranched polysaccharides consisting of a repeating disaccharide unit. The monosaccharide subunits of an glycosaminoglycan have one of their hydroxy groups (commonly but not necessarily in position 2) replaced by an amino group.

As used herein, the term “homopolysaccharide” refers to a polysaccharide which is made up of a single repeating monosaccharide unit (e.g. cellulose is comprised of repeating glucose units).

As used herein, the term “glucan” refers to a polysaccharide of D-glucose monomers. Glucans differ in the type of 0-glycosidic bonds that connect the glucose monomers and the positions on adjacent glucose molecules that are bonded (e.g. cellulose is β-1,4-glucan, curdlan is a β-1,3-glucan, dextran is α-1,6-glucan, glycogen is a α-1,4- and α-1,6-glucan, etc.)

The terms “glucan derivative” or “modified glucan” as used herein to refer to any compound comprising a glucan as a constitutional ingredient, compounds obtained by partially or completely by modifying or chemically altering a glucan, or a glucan in which substituents have been added, regularly or irregularly, onto the monomers (e.g. typically onto or replacing one or more hydroxyl groups of glucose).

The terms “dextran derivative” or “modified dextran” as used herein refers to compounds comprising dextran as a constitutional ingredient, compounds obtained by partially or completely by modifying or chemically altering dextran, or compounds formed by the addition of substituents, regularly or irregularly, onto the monomers of dextran, typically onto or replacing one or more hydroxyl groups.

As used herein, the term “dextran ester” refers to any dextran molecule which has been modified to contain one or more ester groups. Dextran esters typically refer to dextran molecules that have been modified by the replacement of one or more hydroxyl groups with an ester substituent. Dextran esters may occur with differing degree of esterification. Typically, dextran esters have a somewhat regular degree of esterification across all monomer units, however irregularly modified dextrans, in which only a portion of monomers or a single monomer are modified may also be classified as dextran esters.

As used herein, the term “ether of dextran” refers to refers to any dextran molecule which has been modified to contain one or more ether groups. Ethers of dextran typically refer to dextran molecules that have been modified by the replacement of one or more hydroxyl groups with an ether substituent. Ethers of dextran may also occur with differing degree of etherification. Typically, ethers of dextran have a somewhat regular degree of etherification across all monomer units, however irregularly modified dextrans, in which only a portion of monomers or a single monomer are modified may also be classified as ethers of dextran.

As used herein, the term “sulfated dextran derivative” refers to a dextran molecule which has been modified to contain one or more sulfate groups, typically onto or replacing one or more hydroxyl groups. The sulfate modifications may constitute the only modifications to the dextran molecule or may be included with one or more other modifications or substituent-substitutions to the dextran molecule.

### II. ANTISENSE COMPOUNDS

In certain embodiments, the invention provides antisense oligomeric compounds. In such embodiments, the oligomeric compound is complementary to a target nucleic acid. In certain embodiments, a target nucleic acid is an RNA. In certain embodiments, a target nucleic acid is a non-coding RNA. In certain embodiments, a target nucleic acid encodes a protein. In certain embodiments, a target nucleic acid is selected from a mRNA, a pre-mRNA, a microRNA, a non-coding RNA, including small non-coding RNA, and a promoter-directed RNA. In certain embodiments, oligomeric compounds are at least partially complementary to more than one target nucleic acid. For example, oligomeric compounds of the present invention may be microRNA mimics, which typically bind to multiple targets.

Antisense mechanisms include any mechanism involving the hybridization of an oligomeric compound with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, or splicing of the target nucleic acid.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

Antisense mechanisms also include, without limitation, occupancy. In certain embodiments, by binding to a target nucleic acid, an antisense compound modulates a biological function. For example, in certain embodiments, hybridization of an antisense compound prevents splicing of a pre-mRNA. In certain embodiments, hybridization of an antisense compound enhances splicing, for example by recruiting splice factors. In certain embodiments, hybridization of an antisense compound redirects splicing from one splice site to another.

Antisense mechanisms also include, without limitation RNAi mechanisms, which utilize the RISC pathway. Such RNAi mechanisms include, without limitation siRNA, ssRNA and microRNA mechanisms. Such mechanism include creation of a microRNA mimic and/or an anti-microRNA.

Antisense mechanisms also include, without limitation, mechanisms that hybridize or mimic non-coding RNA other than microRNA or mRNA. Such non-coding RNA includes, but is not limited to promoter-directed RNA and short and long RNA that effects transcription or translation of one or more nucleic acids.

In certain embodiments, antisense compounds specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

In certain embodiments, at least a portion of an antisense compound is at least 100%, 99%, 98%, 95%, 90%, 85%, or 80% complementary to a corresponding portion of a target nucleic acid.

In certain embodiments, a portion of an oligomeric compound is 100% identical to the nucleobase sequence of a microRNA, but the entire oligomeric compound is not fully identical to the microRNA. In certain such embodiments, the length of an oligomeric compound having a 100% identical portion is greater than the length of the microRNA. For example, a microRNA mimic consisting of 24 linked nucleosides, where the nucleobases at positions 1 through 23 are each identical to corresponding positions of a microRNA that is 23 nucleobases in length, has a 23 nucleoside portion that is 100% identical to the nucleobase sequence of the microRNA and has approximately 96% overall identity to the nucleobase sequence of the microRNA.

In certain embodiments, the nucleobase sequence of oligomeric compound is fully identical to the nucleobase sequence of a portion of a microRNA. For example, a single-stranded microRNA mimic consisting of 22 linked nucleosides, where the nucleobases of positions 1 through 22 are each identical to a corresponding position of a microRNA that is 23 nucleobases in length, is fully identical to a 22 nucleobase portion of the nucleobase sequence of the microRNA. Such a single-stranded microRNA mimic has approximately 96% overall identity to the nucleobase sequence of the entire microRNA, and has 100% identity to a 22 nucleobase portion of the microRNA.

In certain embodiments, the oligomeric compound is produced by any of a variety of methods. For example, in certain embodiments, the oligomeric compound is produced according to methods described in U.S. Pat. No. 6,465,628, which is incorporated by reference in its entirety.

In certain embodiments, such oligomeric compounds are modified oligonucleotides. In certain embodiments, modified oligonucleotides comprise modified nucleosides. In certain embodiments, modified oligonucleotides of the present invention comprise modified internucleoside linkages. In certain embodiments, modified oligonucleotides of the present invention comprise modified nucleosides and modified internucleoside linkages.

### A. Certain Modified Nucleosides

In certain embodiments, modified oligonucleotides of the present invention comprise modified nucleosides comprising a modified sugar moiety. In certain embodiments, modified oligonucleotides of the present invention comprise modified nucleosides comprising a modified nucleobase. In certain embodiments, modified oligonucleotides of the present invention comprise modified nucleosides comprising a modified sugar moiety and a modified nucleobase.

i. Certain Modified Sugar Moieties

In certain embodiments, the present invention provides modified oligonucleotides comprising one or more nucleosides comprising a modified sugar moiety. In certain embodiments, a modified sugar moiety is a bicyclic sugar moiety. In certain embodiments a modified sugar moiety is a non-bicyclic modified sugar moiety.

Certain modified sugar moiety moieties are known and can be used to alter, typically increase, the affinity of the antisense compound for its target and/or increase nuclease resistance. A representative list of preferred modified sugar moieties includes but is not limited to bicyclic modified sugar moieties (BNA's), including methyleneoxy (4′-CH₂—O-2′) BNA, ethyleneoxy (4′-(CH₂)₂—O-2′) BNA and methyl(methyleneoxy) (4′-C(CH₃)H—O-2′) BNA; substituted sugar moieties, especially 2′-substituted sugar moieties having a 2′-F, 2′-OCH₃ or a 2′-O(CH₂)₂—OCH₃ substituent group; and 4′-thio modified sugar moieties. Sugar moieties can also be replaced with sugar moiety mimetic groups among others. Methods for the preparations of modified sugar moieties are well known to those skilled in the art. Some representative patents and publications that teach the preparation of such modified sugar moieties include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; 6,172,209; 6,271,358; and 6,600,032; and WO 2005/121371.

a. Certain Bicyclic Sugar Moieties

In certain embodiments, the present invention provides modified nucleosides comprising a bicyclic sugar moiety. Examples of bicyclic nucleosides include without limitation nucleosides comprising a bridge between the 4′ and the 2′ ribosyl ring atoms. In certain embodiments, oligomeric compounds provided herein include one or more bicyclic nucleosides wherein the bridge comprises one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya, et al., J. Org. Chem., 2009, 74, 118-134); and 4′-CH₂—C(═CH₂)-2′ (and analogs thereof see published International Application WO 2008/154401, published on Dec. 8, 2008). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226). Certain such sugar moieties have been described. See, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-79 (Jul. 4, 2007); U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181; each of which is incorporated by reference in its entirety.

In certain embodiments, nucleosides comprising a bicyclic sugar moiety have increased affinity for a complementary nucleic acid. In certain embodiments, nucleosides comprising a bicyclic sugar moiety provide resistance to nuclease degradation of an oligonucleotide in which they are incorporated. For example, methyleneoxy (4′-CH₂—O-2′) BNA and other bicyclic sugar moiety analogs display duplex thermal stabilities with complementary DNA and RNA (Tm=+3 to +10° C.), stability towards 3′-exonucleolytic degradation and good solubility properties. Antisense oligonucleotides comprising BNAs have been described (Wahlestedt et al., Proc. Natl. Acad. Sci. U.S.A, 2000, 97, 5633-5638).

Certain bicyclic-sugar moiety containing nucleosides (or BNA nucleosides) comprise a bridge linking the 4′ carbon and the 2′ carbon of the sugar moiety. In certain embodiments, the bridging group is a methyleneoxy (4′-CH₂—O-2′). In certain embodiments, the bridging group is an ethyleneoxy (4′-CH₂CH₂—O-2′) (Singh et al., Chem. Commun., 1998, 4, 455-456: Morita et al., Bioorganic Medicinal Chemistry, 2003, 11, 2211-2226).

In certain embodiments, bicyclic sugar moieties of BNA nucleosides include, but are not limited to, compounds having at least one bridge between the 4′ and the 2′ position of the sugar moiety wherein such bridges independently comprises 1 or from 2 to 4 linked groups independently selected from —[C(Rₐ)(R_{b})]n-, —C(Rₐ)═C(R_{b})—, —C(Ra)═N—, —C(═NRₐ)—, —C(═O)—, —C(═S)—, —O—, —Si(Rₐ)₂—, —S(═O)x-, and —N(R₁)—; wherein:

x is 0, 1, or 2;

n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R₁)—O— or —C(RₐR_{b})—O—N(Rₐ)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(Rₐ)-2′ and 4′-CH₂—N(Rₐ)—O-2′- wherein each Rₐ is, independently, H, a protecting group or C₁-C₁₂ alkyl.

In certain embodiments, bicyclic nucleosides are further defined by isomeric configuration. For example, a nucleoside comprising a 4′-2′ methylenoxy bridge, may be in the α-L configuration or in the β-D configuration. Previously, alpha-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-C(CH₃)H—O-2′) BNA, as depicted below.

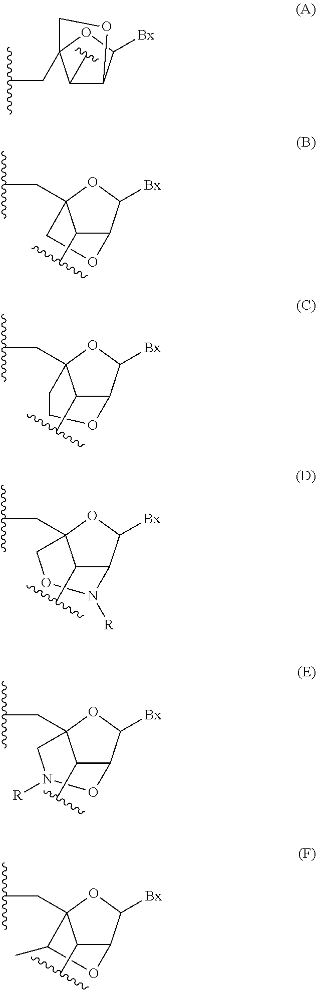

wherein Bx is the base moiety. In certain embodiments, bicyclic nucleosides include, but are not limited to, the structures below:

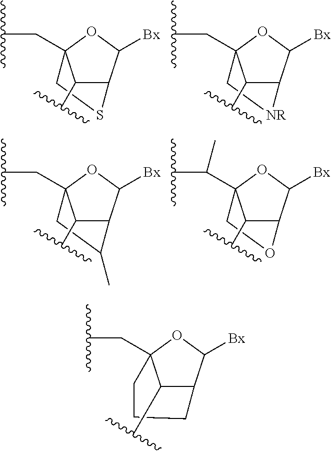

wherein Bx is the base moiety.

In certain embodiments, bicyclic nucleoside having the formula:

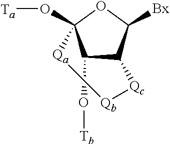

wherein
Bx is a heterocyclic base moiety;

-Qa-Qb-Qc- is —CH₂—N(R_{c})—CH₂—, —C(═O)—N(R_{c})—CH₂—, —CH₂—O—N(R_{c})— or N(R_{c})—O—CH₂—;

R_{c} is C₁-C₁₂ alkyl or an amino protecting group; and
Ta and Tb are each, independently, hydroxyl, a protected hydroxyl, a conjugate group, an activated phosphorus moiety or a covalent attachment to a support medium.

In certain embodiments, bicyclic nucleoside having the formula:

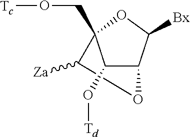

wherein:

Bx is a heterocyclic base moiety;

T_{c} is H or a hydroxyl protecting group;

T_{d} is H, a hydroxyl protecting group or a reactive phosphorus group;

Za is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In one embodiment, each of the substituted groups, is, independently, mono or poly substituted with optionally protected substituent groups independently selected from halogen, oxo, hydroxyl, OJ_{c}, NJ_{c}J_{d}, SJ_{c}, N₃, OC(═X)J_{c}, OC(═X)NJ_{c}J_{d}, NJₑC(═X)NJ_{c}J_{d} and CN, wherein each J_{c}, J_{d} and Jₑ is, independently, H or C₁-C₆ alkyl, and X is O, S or NJ_{c}.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)Jc, and NJeC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In one embodiment, the Zₐ group is C₁-C₆ alkyl substituted with one or more Xx, wherein each Xx is independently OJ_{c}, NJ_{c}J_{d}, SJ_{c}, N₃, OC(═X)J_{c}, OC(═X)NJ_{c}J_{d}, NJₑC(═X)NJ_{c}J_{d} or CN; wherein each J_{c}, J_{d} and Jₑ is, independently, H or C₁-C₆ alkyl, and X is O, S or NJ_{c}. In another embodiment, the Zₐ group is C₁-C₆ alkyl substituted with one or more Xₓ, wherein each Xₓ is independently halo (e.g., fluoro), hydroxyl, alkoxy (e.g., CH₃O—), substituted alkoxy or azido.

In certain embodiments, bicyclic nucleoside having the formula:

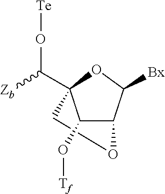

wherein:

Bₓ is a heterocyclic base moiety;

one of Tₑ and T_{f} is H or a hydroxyl protecting group and the other of Tₑ and T_{f} is H, a hydroxyl protecting group or a reactive phosphorus group;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—);

wherein each substituted group is mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ_{f}J_{g}, N₃, COOJ_{f}, CN, O—C(═O)NJ_{f}J_{g}, N(H)C(═NH)NR_{d}Rₑ or N(H)C(═X)N(H)J_{g} wherein X is O or S; and

each J_{f} and J_{g} is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₁-C₆ aminoalkyl, substituted C₁-C₆ aminoalkyl or a protecting group.

In certain embodiments, bicyclic nucleoside having the formula:

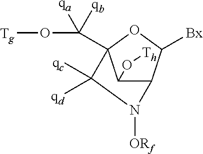

wherein:

Bₓ is a heterocyclic base moiety;

one of T_{g} and Tₕ is H or a hydroxyl protecting group and the other of T_{g} and Tₕ is H, a hydroxyl protecting group or a reactive phosphorus group;

R_{f} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

qa and qb are each independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

qc and qd are each independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

wherein each substituted group is, independently, mono or poly substituted with substituent groups independently selected from halogen, OJₕ, SJₕ, NJₕJᵢ, N₃, COOJₕ, CN, O—C(═O)NJₕJᵢ, N(H)C(═NH)NJₕJᵢ or N(H)C(═X)N(H)Jᵢ wherein X is O or S; and

each Jₕ and Jᵢ is, independently, H, C₁-C₆ alkyl, C₂-C₆ alkenyl, C₂-C₆ alkynyl, C₁-C₆ aminoalkyl or a protecting group.

In certain embodiments, bicyclic nucleoside having the formula:

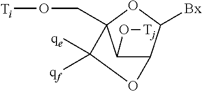

wherein:

Bₓ is a heterocyclic base moiety;

one of Tᵢ and Tⱼ is H or a hydroxyl protecting group and the other of Tᵢ and Tⱼ is H, a hydroxyl protecting group or a reactive phosphorus group;

qe and qf are each, independently, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO2Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qe and qf together are ═C(qg)(qh);

qg and qh are each, independently, H, halogen, C₁-C₁₂ alkyl or substituted C₁-C₁₂ alkyl;

each substituted group is, independently, mono or poly substituted with substituent groups independently selected from halogen, C₁-C₆ alkyl, C₂-C₆ alkenyl, C₂-C₆ alkynyl, OJⱼ, SJⱼ, NJⱼJₖ, N₃, CN, C(═O)OJⱼ, C(═O)NJⱼJₖ, C(═O)Jⱼ, O—C(═O)NJⱼJₖ, N(H)C(═O)NJⱼJₖ or N(H)C(═S)NJⱼJₖ; and

each Jⱼ and Jₖ is, independently, H, C₁-C₆ alkyl, C₂-C₆ alkenyl, C₂-C₆ alkynyl, C₁-C₆ aminoalkyl or a protecting group.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-Amino- and 2′-methylamino-BNA's have been prepared and the thermal stability of their duplexes with complementary RNA and DNA strands has been previously reported.

b. Certain Non-Bicyclic Modified Sugar Moieties

In certain embodiments, the present invention provides modified nucleosides comprising modified sugar moieties that are not bicyclic sugar moieties. Certain such modified nucleosides are known. In certain embodiments, the sugar ring of a nucleoside may be modified at any position.

Examples of sugar modifications useful in this invention include, but are not limited to compounds comprising a sugar substituent group selected from: OH, F, O-alkyl, S-alkyl, N-alkyl, or O-alkyl-O-alkyl, wherein the alkyl, alkenyl and alkynyl may be substituted or unsubstituted C₁ to C₁₀ alkyl or C₂ to C₁₀ alkenyl and alkynyl. In certain such embodiments, such substituents are at the 2′ position of the sugar.

In certain embodiments, modified nucleosides comprise a substituent at the 2′ position of the sugar. In certain embodiments, such substituents are selected from among: a halide, including, but not limited to F, allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rm)(Rn), or O—CH₂—C(═O)—N(Rm)(Rn), where each Rm and Rn is, independently, H or substituted or unsubstituted C₁-C₁₀ alkyl.

In certain embodiments, modified nucleosides suitable for use in the present invention are: 2-methoxyethoxy, 2′-O-methyl (2′-O—CH₃), 2′-fluoro (2′-F).

In certain embodiments, modified nucleosides having a substituent group at the 2′-position selected from: O[(CH₂)nO]mCH₃, O(CH₂)nNH₂, O(CH₂)nCH₃, O(CH₂)nONH₂, OCH₂C(═O)N(H)CH₃, and O(CH₂)nON[(CH₂)nCH₃]₂, where n and m are from 1 to about 10. Other 2′-sugar substituent groups include: C₁ to C₁₀ alkyl, substituted alkyl, alkenyl, alkynyl, alkaryl, aralkyl, O-alkaryl or O-aralkyl, SH, SCH₃, OCN, C₁, Bᵣ, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties.

In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

In certain embodiments, 2′-Sugar substituent groups are in either the arabino (up) position or ribo (down) position. In certain such embodiments, a 2′-arabino modification is 2′-F arabino (FANA). Similar modifications can also be made at other positions on the sugar, particularly the 3′ position of the sugar on a 3′ terminal nucleoside or in 2′-5′ linked oligonucleotides and the 5′ position of 5′ terminal nucleotide.

In certain embodiments, sugar moieties comprise sugar surrogates, in which the nucleoside furanose ring of an unmodified nucleoside is replaced with a non-furanose (or 4′-substituted furanose) group having another structure such as a different ring system or open system. Such structures can be as simple as a six membered ring as opposed to the five membered furanose ring or can be more complicated as is the case with the non-ring system used in peptide nucleic acid. The term is meant to include replacement of the sugar group with all manner of sugar surrogates know in the art and includes without limitation sugar surrogate groups such as morpholinos, cyclohexenyls and cyclohexitols. In most monomer subunits having a sugar surrogate group the heterocyclic base moiety is generally maintained to permit hybridization.

In certain embodiments, nucleosides having sugar surrogate groups include without limitation, replacement of the ribosyl ring with a surrogate ring system such as a tetrahydropyranyl ring system (also referred to as hexitol) as illustrated below:

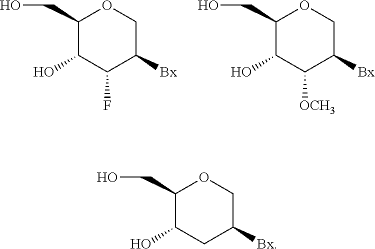

Many other monocyclic, bicyclic and tricyclic ring systems are known in the art and are suitable as sugar surrogates that can be used to modify nucleosides for incorporation into oligomeric compounds as provided herein (see for example review article: Leumann, Christian J.). Such ring systems can undergo various additional substitutions to further enhance their activity.

Some representative U.S. patents that teach the preparation of such modified sugars include without limitation, U.S.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005 certain of which are commonly owned with the instant application, and each of which is herein incorporated by reference in its entirety.

In certain embodiments, nucleosides suitable for use in the present invention have sugar surrogates such as cyclobutyl in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

In certain embodiments, the present invention provides nucleosides comprising a modification at the 2′-position of the sugar. In certain embodiments, the invention provides nucleosides comprising a modification at the 5′-position of the sugar. In certain embodiments, the invention provides nucleosides comprising modifications at the 2′-position and the 5′-position of the sugar. In certain embodiments, modified nucleosides may be useful for incorporation into oligonucleotides. In certain embodiment, modified nucleosides are incorporated into oligonucleosides at the 5′-end of the oligonucleotide.

2. Certain Modified Nucleobases

In certain embodiments, nucleosides of the present invention comprise unmodified nucleobases. In certain embodiments, nucleosides of the present invention comprise modified nucleobases.

In certain embodiments, nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Certain modified nucleobases are disclosed in, for example, Swayze, E. E. and Bhat, B., The medicinal Chemistry of Oligonucleotides in Antisense Drug Technology, Chapter 6, pages 143-182 (Crooke, S. T., ed., 2008); U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-₂, N-₆ and O-₆ substituted purines, including 2 aminopropyladenine, 5-propynyluracil and 5-propynylcytosine.

In certain embodiments, nucleobases comprise polycyclic heterocyclic compounds in place of one or more heterocyclic base moieties of a nucleobase. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs.

Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). When incorporated into oligonucleotides, these base modifications have been shown to hybridize with complementary guanine and the latter was also shown to hybridize with adenine and to enhance helical thermal stability by extended stacking interactions (also see U.S. Patent Application Publication 20030207804 and U.S. Patent Application Publication 20030175906, both of which are incorporated herein by reference in their entirety).

Helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTm of up to 180 relative to 5-methyl cytosine (dC₅me), which is the highest known affinity enhancement for a single modification. On the other hand, the gain in helical stability does not compromise the specificity of the oligonucleotides. The Tm data indicate an even greater discrimination between the perfect match and mismatched sequences compared to dC₅me. It was suggested that the tethered amino group serves as an additional hydrogen bond donor to interact with the Hoogsteen face, namely the O₆, of a complementary guanine thereby forming 4 hydrogen bonds. This means that the increased affinity of G-clamp is mediated by the combination of extended base stacking and additional specific hydrogen bonding.

Tricyclic heterocyclic compounds and methods of using them that are amenable to the present invention are disclosed in U.S. Pat. No. 6,028,183, and U.S. Pat. No. 6,007,992, the contents of both are incorporated herein in their entirety.

The enhanced binding affinity of the phenoxazine derivatives together with their sequence specificity makes them valuable nucleobase analogs for the development of more potent antisense-based drugs. The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

Modified polycyclic heterocyclic compounds useful as heterocyclic bases are disclosed in but not limited to, the above noted U.S. Pat. No. 3,687,808, as well as U.S.: 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Patent Application Publication 20030158403, each of which is incorporated herein by reference in its entirety.

3. Certain Internucleoside Linkages

In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligonucleotides having non-phosphorus internucleoside linking groups may be referred to as oligonucleosides. Modified linkages, compared to natural phosphodiester linkages, can be used to alter, typically increase, nuclease resistance of the oligomeric compound. In certain embodiments, internucleoside linkages having a chiral atom can be prepared a racemic mixtures, as separate enantomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantomers, diastereomers, and other stereoisomeric configurations that may be defined, in terms of absolute stereochemistry, as (R) or (S), α or β such as for sugar anomers, or as (D) or (L) such as for amino acids et al. Included in the antisense compounds provided herein are all such possible isomers, as well as their racemic and optically pure forms.

### B. Lengths of Oligomeric Compounds

In certain embodiments, the invention provides oligomeric compounds comprising oligonucleotides. In certain embodiments, the present invention provides oligomeric compounds including oligonucleotides of any of a variety of ranges of lengths. In certain embodiments, the invention provides oligomeric compounds comprising oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≦Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide consisting of 8-30 nucleosides excludes oligonucleotides having 31 nucleosides, but, unless otherwise indicated, such an oligonucleotide may further comprise, for example one or more conjugates, terminal groups, or other substituents. In certain embodiments, terminal groups include, but are not limited to, terminal group nucleosides. In such embodiments, the terminal group nucleosides are differently modified than the terminal nucleoside of the oligonucleotide, thus distinguishing such terminal group nucleosides from the nucleosides of the oligonucleotide.

Those skilled in the art, having possession of the present disclosure will be able to prepare oligomeric compounds, comprising a contiguous sequence of linked monomer subunits, of essentially any viable length to practice the methods disclosed herein.

In certain embodiments, antisense compounds are gapmers. In certain embodiments, antisense compounds are uniformly modified. In certain embodiments, antisense compounds comprise a region of alternating modifications.

### E. Conjugates

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

2. Terminal Groups

In certain embodiments, oligomeric compounds comprise terminal groups at one or both ends. In certain embodiments, a terminal group may comprise any of the conjugate groups discussed above. In certain embodiments, terminal groups may comprise additional nucleosides and/or inverted abasic nucleosides. In certain embodiments, a terminal group is a stabilizing group.

In certain embodiments, oligomeric compounds comprise one or more terminal stabilizing group that enhances properties such as for example nuclease stability. Included in stabilizing groups are cap structures. The terms “cap structure” or “terminal cap moiety,” as used herein, refer to chemical modifications, which can be attached to one or both of the termini of an oligomeric compound. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270).

Particularly suitable 3′-cap structures of the present invention include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxy-pentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925 and Published U.S. Patent Application Publication No. US 2005/0020525 published on Jan. 27, 2005). Further 3′ and 5′-stabilizing groups that can be used to cap one or both ends of an oligomeric compound to impart nuclease stability include those disclosed in WO 03/004602.

3. Additional Nucleosides

In certain embodiments, one or more additional nucleosides is added to one or both terminal ends of an oligonucleotide or an oligomeric compound. In a double-stranded compound, such additional nucleosides are terminal (3′ and/or 5′) overhangs. In the setting of double-stranded antisense compounds, such additional nucleosides may or may not be complementary to a target nucleic acid.

In a single-stranded antisense oligomeric compound, additional nucleosides are typically non-hybridizing terminal nucleosides. The additional nucleosides are typically added to provide a desired property other than hybridization with target nucleic acid. Nonetheless, the target may have complementary bases at the positions corresponding with the additional nucleosides. Whether by design or accident, such complementarity of one or more additional nucleosides does not alter their designation as additional. In certain embodiments, the bases of additional nucleosides are each selected from adenine (A), uracil (U), and thymine (T). In certain embodiments, the additional nucleosides each comprise adenine (A) or uracil (U) nucleobases. In certain embodiments, the additional nucleosides each comprise adenine (A) nucleobases. In certain embodiments, the additional nucleosides each comprise uracil (U). In certain embodiments, the additional nucleosides each comprise thymine (T). In certain embodiments, additional nucleosides comprise guanine nucleobases. In certain embodiments, additional nucleosides comprise cytosine nucleobases.

In certain embodiments, additional nucleosides are sugar modified. In certain such embodiments, such additional nucleosides are 2′-modified. In certain embodiments, additional nucleosides are 2′-MOE modified. In certain embodiments, additional nucleosides are MOE adenine (MOE A) nucleosides. In certain embodiments, additional nucleosides are MOE uracil (MOE U) nucleosides. In certain embodiments, additional nucleosides are MOE thymine (MOE T) nucleosides. In certain embodiments, 1-5 such additional MOE A and/or MOE U and/or MOE T nucleosides are added to the 3′-end of an oligomeric compound.

In certain embodiments, additional nucleosides are BNAs. In certain embodiments, additional nucleosides are LNA nucleosides. In certain embodiments, additional nucleosides are LNA adenine (LNA A) nucleosides. In certain embodiments, additional nucleosides are LNA uracil (LNA U) nucleosides. In certain embodiments, additional nucleosides are LNA thymine (LNA T) nucleosides. In certain embodiments, 1-5 such additional LNA A and/or LNA U and/or LNA T nucleosides are added to the 3′-end of an oligomeric compound.

In certain embodiments having two or more additional nucleosides, the two or more additional nucleosides all have the same modification type and the same base. In certain embodiments having two or more additional nucleosides, the additional nucleosides differ from one another by modification and/or base.

### III EXCIPIENTS

Antisense compounds, including, but not limited to those described above may be combined with one or more antisense excipient of the present invention.

In some embodiments of the present invention, the excipient is a polyanion (e.g. deoxyribonucleic acid, ribonucleic acid, polysaccharide, polypeptide, etc.). In some embodiments, the excipient of the present invention is a polyanion polymer. In some embodiments, the excipient is an anionic polymer. In some embodiments, the excipient is a nucleic acid molecule. In some embodiments, the excipient is a non-antisense nucleic acid molecule. In some embodiments, the excipient is not a nucleic acid molecule. In some embodiments, the excipient is a glycan (e.g. polysaccharide, oligosaccharide (e.g. oligofructose, galactooligosaccharide, etc.), the carbohydrate portion of a glycoconjugate (e.g. glycoprotein, glycolipid, proteoglycan, etc.), etc.). In some embodiments, the excipient is a polysaccharide (e.g. repeating monosaccharide, repeating disaccharide, homopolysaccharide, heteropolysaccharide, hyaluronate, chondroitin, amylase, glycogen, amylopectin, laminarin, xylan, mannan, fucoidan, galactomannan, etc.). In some embodiments, the excipient is not heparin. In some embodiments, the excipient is not a glycosaminoglycan. In some embodiments, the excipient is a homopolysaccharide (e.g. cellulose, glycogen, etc.). In some embodiments, the excipient is a branched polysaccharide (e.g. dextran, scleroglucan, etc.). In some embodiments, the excipient is a chemically or enzymatically modified glucan (e.g. cellulose, curdlan, dextran, glycogen, laminarian, lentinian, lichenin, pleuran, pullulan, starch, zymosan, etc.) In some embodiments, the excipient of the present invention is a glucan derivative (e.g. lauryldextran, glucan esters, lami-naribiose, cellobiose, nigerose, laminaritriose, laminaritetrose and laminaripentos, sulfated glucans, α-glucan derivative, β-glucan derivative etc.). In some embodiments, the excipient is a branched glucan derivative. In some embodiments, the excipient is a dextran derivative or modified dextran (e.g. dextran phenyl carbonate, dextran ethyl carbonate, dextran tributyrate, dextran tripropionate, dextran tributyrate, dextran benzyl ether, dextran triacetate, dextran triheptanoate, dextran butyl carbamate, etc.). In some embodiments, the excipient is a dextran ester (e.g. caproyldextran, stearyldextran, lauryldextran, acetyldextran, etc.). In some embodiments, the excipient is an ether of dextran (e.g. sulfopropyl ether of dextran, phosphonomethyl ether of dextran, mercaptoethyl ether of dextran, 3-chloro-2-hydroxypropyl ether of dextran, cyanoethyl ether of dextran, 2-(3′-amino-4′-methoxyphenyl)-sulfonylethyl ether of dextran, etc.). In some embodiments, the excipient is a sulfur-containing dextran derivative (e.g. sulfopropyl-dextran, mercaptoethyl-dextran, 2-(3′-amino-4′-methoxyphenyl)-sulfonylethyl-dextran, etc.). In some embodiments, the excipient is a sulfur-containing dextran ester. In some embodiments, the excipient is a sulfated dextran derivative (e.g. carboxymethylated sulfated dextran, etc.). In some embodiments, the excipient is a sulfate containing molecule (e.g. dextran, sulfated polyvinyl alcohol (PVAS), polyvinyl sulfate (PVS), PRO-2000, sulfated copolymers of acrylic acid and vinyl alcohol (PAVAS), etc.). In some embodiments the excipient is dextran sulfate. In some embodiments, the excipient comprises a mixture of two or more of the above agents.

In some embodiments, excipients are formulated with therapeutic or research oligonucleotides. In other embodiments, excipients are formulated independently from the antisense or research oligonucleotides. In some embodiments, kits are provided containing separately formulated excipient and oligonucleotide, for example, each in their own container. In some embodiments, excipients are formulated in dosage form for either single or multiple administrations to subjects, tissues, or cells. In some embodiments the concentration for in vivo administration is from 0.1 mM to 50 mM. The concentration and dose may be optimized, as desired, based on the particular application and considering characteristics of the treated subject, such as age, weight, gender, and the like.

In some embodiments, the amount and type of excipient is selected so as to increase cellular uptake of the oligonucleotide via a productive mechanism. Examples 1-3 below provide exemplary assays for identifying and selecting appropriate conditions. Thus, in some embodiments, the present invention provides excipients and formulations designed to take advantage of productive uptake and/or avoid unproductive accumulation. In certain embodiments, the invention provides formulations of antisense oligomeric compounds for administration comprising excipients that saturate the unproductive mechanism. Such excipients may be administered separately or together with an antisense oligomeric compound. If administered separately they may administered through the same route of administration or through different routes of administration. They may be administered at the same time or at different times. In certain embodiments, the excipient is first administered and the antisense oligomeric compound is later administered. Such administration includes, but is not limited to administration to an animal, including, but not limited to a human.

The excipients of the present invention find use in a wide variety of applications, including research uses and therapeutic uses to treat or prevent diseases (e.g., infectious diseases and non-infectious diseases such as cancer, heart disease, genetic diseases, and the like), illnesses (e.g., lethargy, depression, anorexia, sleepiness, hyperalgesia, and the like), disorders (e.g., mental disorders, physical disorder, genetic disorders, behavioral disorder, functional disorders, and the like), and other medical conditions, as well as to provide biological benefits to otherwise healthy individuals.

### G. Kits, Research Reagents and Diagnostics

The cells and assays provided herein can be utilized as research reagents and kits. For use in kits, either alone or in combination with other compounds or reagents, cells and oligomeric compounds of the present invention can be used as tools useful for studying uptake and intracellular trafficking of oligomeric compounds. In some embodiments, the kits of the present invention provide one or more components sufficient, necessary, or useful for carrying out any of the methods described herein.

Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

The nucleobase sequences set forth herein, including, but not limited to those found in the Examples and in the sequence listing, are independent of any modification to the nucleic acid. As such, nucleic acids defined by a SEQ ID NO may comprise, independently, one or more modifications to one or more sugar moiety, to one or more nucleobase, and/or to one or more internucleoside linkage.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is somewhat arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, unless otherwise indicated, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified bases, such as “AT^{me}CGAUCG,” wherein ^{me}C indicates a cytosine base comprising a methyl group at the 5-position.

### EXAMPLES

The following examples are provided in order to demonstrate and further illustrate certain preferred embodiments and aspects of the present invention and are not to be construed as limiting the scope thereof.

### Example 1

### In Culture Evaluation of Excipients

Excipients can be screened for activity using any of a variety of methods. This example provides exemplary methods illustrated using PTEN as a biological target testing in culture.

MHT cells

Transgenic mice engineered to express the SV40 large T antigen (SV40 t/T mice) under the control of the liver-specific C-reactive protein promoter are a source of transformed cells. The expression of SV40 large T antigen can be transiently induced by injection of bacterial lipopolysaccacharide. The cells can be isolated from the livers of the transgenic mice. The cells are herein referred to as Mouse Hepatocyte SV40 T-Antigen expressing cells, or MHT cells.

Transgenic mice are anesthetized, and perfusion into the portal vein of the liver is performed to introduce collagenase into the liver tissue. The livers are isolated from the livers of SV40 t/T mice, the tissue is gently homogenized and a hepatocyte cell fraction is isolated. The cells are then placed in 12-well plates, with and without a collagen coating. The culture medium is either DMEM containing 10% fetal bovine serum (FBS) or William's Medium E containing 10% FBS, 10 mM HEPES, and glutamine. The culture medium is changed every 3 days, and any growing cells are transferred to 6-well culture plates for continued culture and expansion. Distinct populations of cells are present after approximately one month of culture. SV40 mRNA expression is monitored using real-time PCR with primers specific for SV40 mRNA; cells expressing the SV40 mRNA are identified as MHT cells.

Cells are diluted and placed into wells of a 96-well collagen-coated culture plate such that no more than one cell is in a single well. Cells are allowed to expand. Several clones are selected and found to express SV40 mRNA, as measured by real-time PCR.

Uptake of Oligomeric Compounds into MHT Cells

Antisense oligonucleotides (ASOs) may be administered to cells either via transfection reagent or with transfection reagent. Where transfection reagents are desired, a lipofection:oligomeric compound ratio of 3 ug/mL:100 nM may be used with or without excipient. Oligomeric compound concentrations are 25, 50, 100, or 200 nM. An untreated sample serves as a control. In the absence of transfection reagent, cells are plated in collagen-coated 96-well plates at a density of 5000 cells per well. After one day in culture, cells are washed with phosphate-buffered saline (PBS), then overlaid with William's E Medium containing 1% FBS, 0.1% bovine serum albumin (BSA), and the desired concentration of oligomeric compound (either 1 uM or 4 uM), with or without excipient. Untreated cells served as a control. After 48 hours, RNA is isolated from the cells and SV40 mRNA is measured by real-time PCR.

### Competition Assay to Assess the Relative Uptake of Oligomeric Compounds

In certain embodiments, the invention provides a competition assay useful for assessing the uptake of oligomeric compounds. In certain embodiments, the competition assay is useful for assessing the relative uptake of oligomeric compounds. The competition assay employs a competitor oligomeric compound and a reporter oligomeric compound. In certain embodiments, the concentration of the competitor oligomeric compound remains constant while the concentration of the reporter oligomeric compound is varied.

An oligomeric compound complementary to SR-B1 mRNA (SR-B1 oligo) is used as a reporter oligomeric compound. An oligomeric compound complementary to PTEN (PTEN oligo) is used as a competitor oligomeric compound. Cultured MHT cells are cultured in serum-containing medium.

One MHT culture receives increasing concentrations of SR-B1 oligo: 10, 100, 1000, and 10000 nM. One day after addition of the oligonucleotides, RNA is isolated from the cells. Real-time PCR is used to assess SR-B1 mRNA levels.

### Localization of Oligomeric Compounds in Cells

The localization of oligomeric compounds in MHT cells is compared following introduction of oligomeric compounds into the cell culture medium. Cells are treated and the following day, the cells are washed and then fixed with 4% formaldehyde for 15 minutes. Fluorescence microscopy is used to identify the cellular location and/or accumulation of oligomeric compounds, for example, in the presence and absence of excipients.

### Oligomeric Compound Uptake in the Presence of Excipients

The effects of the excipients on the uptake of oligomeric compounds can be assessed in vitro in MHT cells. To assess the effects of excipient on oligomeric compound activity, MHT cells are treated with increasing concentrations of SR-B1 oligo in the presence and absence of excipient. One sample includes no excipient and additional samples are treated with SR-B1 oligo and various concentrations of excipient. After 24 hours, SR-B1 mRNA levels are quantitated by real-time PCR to determine the effect of the excipient on ASO activity.

### Example 2

### In Vivo Evaluation of Excipients

Excipients can be screened for in vivo activity using any of a variety of methods. This example provides exemplary methods illustrated using PTEN as a biological target.

Antisense Oligonucleotide (ASO) Synthesis and Chemistry.

2′-O-(methoxyethyl) modified antisense phosphorothioate oligonucleotides (2′-MOE ASOs) are synthesized as described previously (Baker et al., J. Biol. Chem., 272:11994 (1997), herein incorporated by reference in its entirety). ASOs used are 20 nucleotides in length and contain 2′-MOE modifications at the terminal residues at both the 5′ and 3′ ends of the molecule with at least 9 contiguous deoxy-nucleotide residues in the center. The sequences for the ASOs are as follows: ISIS 116847, 5′-CTGCT agcctctgga TTTGA-3′ (active anti-PTEN sequence)(SEQ ID NO: 1) and ISIS 13920, 5′-TCC gtcatcgct CCTCAGGG-3′ (nonsense sequence)(SEQ ID NO: 2), where the bolded/italicized nucleotides are 2′-MOE modified and the “middle”, lower case nucleotides are -deoxy. All of the cytosines in both sequences are methylated at the C-5 position.

In Vivo Mouse Dosing

Balb/C male mice weighing between 20 and 25 g are purchased from Charles River Laboratories (Wilmington, Mass.) and housed 5 per cage in shoe box cages. Mice are maintained on feed and water ad libitum. All animal studies are conducted utilizing protocols and methods approved by the Institutional Animal Care and Use Committee (IACUC) and carried out in accordance with the Guide for the Care and Use of Laboratory Animals adopted and promulgated by the U.S. National Institutes of Health.

Intravenous (i.v.) injection (tail vein) or subcutaneous (s.c.) bolus injection (sub-scapular) are use. Subcutaneous (s.c.) infusions are accomplished using implanted Alzet minipumps (Durect Corp., Cupertino, Calif.) that provided either 24 hr, 72 hr or 168 hr (1 week) continuous infusion. Dose volumes are less than or equal to 10 mL/kg (approximately 200 to 250 uL per mouse).

All dosing studies involve a single dose injection or infusion of ISIS 116847 ASO in saline solution at a dose of 0 (saline control), 15, 30, 60 or 100 mg/kg. ISIS 13920 doses of 15, 30, 60, 90 and 120 mg/kg are co-infused with a dose of 30 mg/kg ISIS 116847 ASO to characterize the effects of direct competition using an inactive, nonsense oligonucleotide (ISIS 13920). ISIS 116847 is also administered as a bolus s.c. injection 0 (immediately after) and 8 hours after a 24-hr infusion of ISIS 13920 oligonucleotide in saline solution to test competition following completion of ISIS 13920 distribution.

Excipient is administered in one or more different doses from 1 nM to 1 mM via i.v. or s.c. One group of animals receives excipient mixed with the ASO. Other animal groups receive excipient prior to ASO administration at time intervals of 30 minutes, up to 24 hours. Control groups are administered excipient only and ASO only. Where two or more different excipients are tested, they may be provided in different doses or timing, as desired.

Plasma is collected and frozen at −70° C. or less for determination of ASO concentration at previously determined maximal concentration time points (immediately after i.v. injection, 0.5 hr after s.c. injection, and approximately 6 hr for all 24 hr infusions). Urine is collected on ice for measuring oligonucleotide excretion over the 24-hr period beginning with the start of dosing. Urine is stored at −70° C. or less until analyzed. Liver and kidneys are collected at sacrifice approximately 72 hr after dosing is completed. A portion of each liver is homogenized in guanidine isothiocyanate buffer for mRNA extraction and RT-PCR analysis of PTEN mRNA. The remaining liver and one kidney are flash frozen and stored at −70° C. or less until assayed for oligonucleotide concentration.

Quantitative Reverse Transcription-PCR

Tissues are homogenized in guanidine isothyocyanate buffer (4 M guanidine isothiocyanate, 25 mM EDTA, 1 M β-mercaptoethanol, 50 mM Tris-HCl, pH 6) immediately following sacrifice. RNA is extracted using RNeasy columns (Qiagen) according to manufacturer's protocol. RNA is eluted from the columns with water. RNA samples are analyzed by fluorescence-based quantitative RT-PCR using an Applied Biosystems 7700 sequence detector. Levels of target RNAs as well as those of cyclophilin A, a housekeeping gene, are determined. Target RNA levels were normalized to cyclophilin levels for each RNA sample. Primers used for determination of PTEN RNA level are as follows: FP 5′ ATGACAATCATGTTGCAGCAATTC 3′ (SEQ ID NO: 3), RP 5′ CGATGCAATAAATATGCACAAATCA 3′ (SEQ ID NO: 4), and PR 5′ 6FAM-CTGTAAAGCTGGAAAGGGACGGACTGGT-TAMRA 3′(SEQ ID NO 5). Primers used for determination of cyclophilin A RNA level are as follows: FP 5′ TCGCCGCTTGCTGCA 3′(SEQ ID NO: 6), RP 5′ ATCGGCCGTGATGTCGA 3′(SEQ ID NO: 7), and PR 5′ 6FAM-CCATGGTCAACCCCACCGTGTTC-TAMRA 3′(SEQ ID NO: 8).

Bioanalytical Methods

Plasma, urine and tissue ASO concentrations are measured using capillary gel electrophoresis (CGE) with UV detection at 260 nm. ASO from plasma and urine are extracted by solid phase extraction methods (SPE) followed by desalting by dialysis prior to electrokinetic introduction of the extracts on column. Tissues are weighed, minced and spiked with a known amount of internal standard prior to homogenization. Tissue homogenates are initially extracted using phenol:chloroform:isoamyl alcohol. Additional sample cleanup varies dependent on the matrix, but generally involved solid phase extraction (SPE) followed by dialysis prior to electrokinetic introduction of analytes from the extract on a Beckman PACE 5000 capillary electrophoresis (CE) instrument (source).

For competition studies, a sequence specific hybridization ELISA method is used as to determine concentrations of ISIS 116847 and ISIS 13920 separately.

Data Analysis

The effect of excipient is determined by comparing mRNA levels and ASO concentrations in the presence and absence of excipient at the different dosing and timing regimens. Excipients that enhance the activity of the ASO are selected for further use and/or testing.

The data may be analyzed to determine the ability of the excipient to increase productive uptake of the ASO. While the present invention is not limited to any particular mechanism of action, and an understanding of the mechanism of action necessary to practice the present invention, it is contemplated that excipients find use in directing ASOs to productive uptake to enhance activity. Oligonucleotides are not expected to passively diffuse across lipid bilayer membranes but rather are thought to enter cells by a series of protein binding interactions that ultimately result in binding to the target mRNA. Following parenteral administration, protein interactions begin in the blood plasma where phosphorothioate oligonucleotides bind to primarily albumin and α2-macroglobulin with the bound fraction exceeding 85% in mouse plasma and greater than 90% in monkey and man. Blood plasma then bathes multiple organs and cells and the oligonucleotides are observed associated to cell surface proteins and extracellular matrix by 1 hr after parenteral administration. While the specifics of the pathways or transporters involved in the process are not yet fully elucidated, it is clear that oligonucleotide appears in the cytoplasm of many cells within a very short time after administration including Kupffer cells and sinusoidal endothelial cells in the liver, and renal proximal epithelial cells in the kidney. Since the ultimate intracellular disposition of oligonucleotides is believed to be a process of protein binding interactions leading from high capacity low affinity proteins in plasma to higher affinity binding in organs of disposition, one would expect that uptake kinetics would be saturable and nonlinear. It has been demonstrated that uptake of ISIS 116847 in whole liver of mice was favored at lower plasma concentrations, consistent with saturable, nonlinear uptake. Reduced urinary excretion also appears to play at least some role in the improvement of whole organ uptake at lower doses and plasma concentrations. Unexpectedly, however, an increase in liver drug concentration due to slower infusion did not provide improved pharmacodynamics at the lowest doses, suggesting that the low plasma concentration-favored an uptake pathway that is, at least in part, a non-productive pathway. When co-administered with a nonsense oligonucleotide that would be expected to compete for an uptake process, the uptake of the active ASO was decreased while activity of the active antisense oligonucleotide, ISIS 116847, was enhanced. These data taken together suggest that there may be a second cellular uptake pathway that is less saturable (lower affinity) that can be favored by competing for the nonproductive pathway (high affinity and saturable). Thus, it is contemplated that there are at least two uptake pathways: the first pathway that is preferred at low plasma concentrations, is thus saturable, but results in sequestration of nonproductive ASO in the cells; the second pathway that is accessed upon saturation of the first pathway and optimized by competition with the higher affinity but nonproductive first pathway. Blocking or saturating the nonproductive high affinity and saturable pathway or by optimizing binding to the currently lower affinity but more productive uptake pathway provide the opportunities for improved ASO pharmacokinetics. Excipients are selected based on their ability to improve ASO activity via these or other mechanisms.

### Example 3

### Dextran Sulfate as an Antisense Excipient

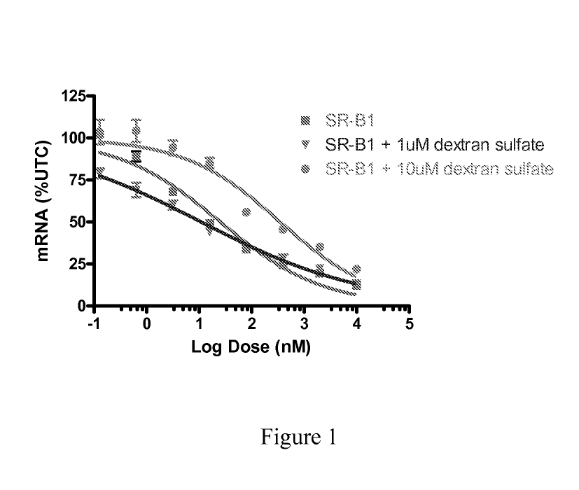

During development of embodiments of the present invention, the effect of antisense oligonucleotides on cellular mRNA levels was tested in the presence and absence of the excipient dextran sulfate (SEE FIG. 1). Antisense oligo SR-B1 was administered to cells at concentrations ranging from 100 pM-10 μM, in the presence (1 μM or 10 μM) or absence of dextran sulfate. Cellular mRNA levels were subsequently measured and compared to the mRNA levels of untreated cells (UTC). High concentrations of dextan sulfate (10 μM dextran sulfate) inhibited mRNA target reduction (SEE FIG. 1) as well as antisense oligo uptake. However, at lower concentration (e.g., 1 μM dextran sulfate), dextran sulfate inhibited antisense oligo uptake without inhibiting target reduction (SEE FIG. 1).

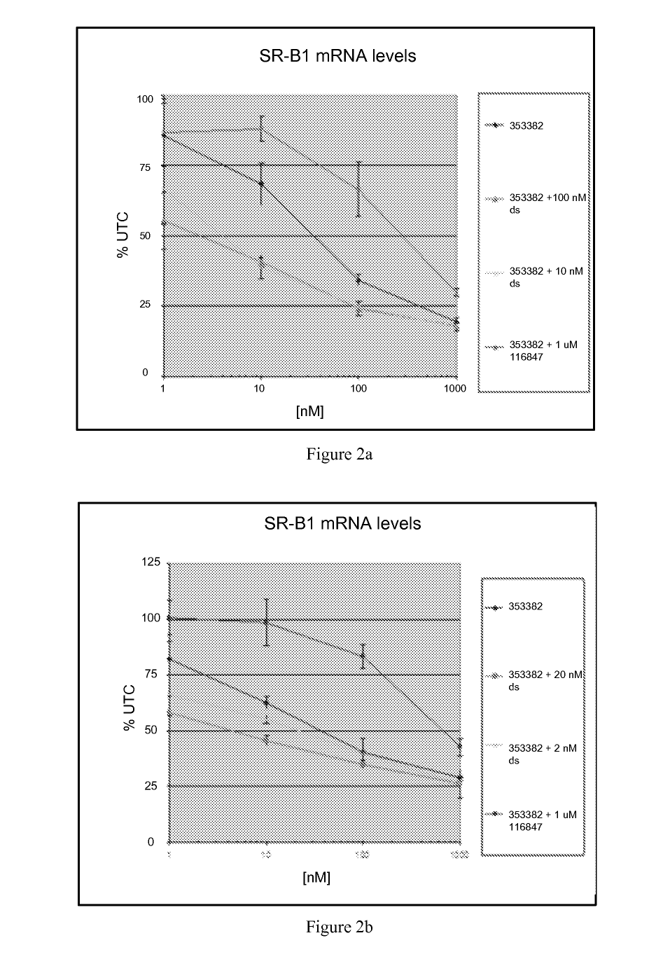

The in vivo effects of dextran sulfate on the efficiency of antisense oligo effects were measured in mice. Antisense oligo (353382) was administered to mice at concentrations ranging from 1 nm to 1000 nm. Antisense oligo was administered with 0 nm, 2 nm, 10 nm, 20 nm, or 100 nm dextran sulfate. Even at the lowest concentrations tested, dextran sulfate increased the antisense oligo effect, evidenced by a decrease in mRNA levels compared to untreated mice, or mice treated with antisense oligo without dextran sulfate (SEE FIGS. 2A and 2B).

1-63. (canceled)

64. A pharmaceutical composition comprising an antisense oligonucleotide; at least one excipient, and purified water or saline solution, wherein at least one excipient is an oligosaccharide or polysaccharide.

65. The pharmaceutical composition of claim 64, wherein at least one excipient is a branched oligosaccharide or branched polysaccharide.

66. The pharmaceutical composition of claim 64, wherein at least one excipient is a glucan or glucan derivative.

67. The pharmaceutical composition of claim 66, wherein at least one excipient is a dextran or dextran derivative.

68. The pharmaceutical composition of claim 67, wherein at least one excipient is selected from: dextran phenyl carbonate, dextran ethyl carbonate, dextran tributyrate, dextran tripropionate, dextran tributyrate, dextran benzyl ether, dextran triacetate, dextran triheptanoate, dextran butyl carbamate.

69. The pharmaceutical composition of claim 67, wherein at least one excipient is selected from a dextran ester, caproyldextran, stearyldextran, lauryldextran, and acetyldextran.

70. The pharmaceutical composition of claim 67, wherein at least one excipient is an ether of dextran.

71. The pharmaceutical composition of claim 70, wherein at least one excipient is sulfopropyl ether of dextran, phosphonomethyl ether of dextran, mercaptoethyl ether of dextran, 3-chloro-2-hydroxypropyl ether of dextran, cyanoethyl ether of dextran, or 2-(3′-amino-4′-methoxyphenyl)-sulfonylethyl ether of dextran.

72. The pharmaceutical composition of claim 67, wherein at least one excipient is dextran sulfate.

73. The pharmaceutical composition of any of claim 67, wherein at least one excipient is a sulfated dextran derivative.

74. The pharmaceutical composition of claim 67, wherein at least one excipient is selected from: dextran, sulfated polyvinyl alcohol (PVAS), polyvinyl sulfate (PVS), PRO-2000, sulfated copolymers of acrylic acid and vinyl alcohol (PAVAS).

75. The pharmaceutical composition of claim 63, wherein the antisense oligonucleotide is complementary to a target nucleic acid selected from: a mRNA, a pre-mRNA, a microRNA, and a non-coding RNA.

76. A method comprising administering to a subject a pharmaceutical composition according to claim 64.

77. The method of claim 76, wherein the modulating effect of the antisense compound is at least 1.125 greater than the modulating effect of administering the same composition without the excipient.

78. A method comprising first administering to a subject an oligosaccharide or polysaccharide and then administering to the subject an antisense compound.

79. The method of claim 78, wherein the oligosaccharide or polysaccharide is a dextran or dextran derivative.

80. The method of claim 78, wherein the modulating effect of the antisense compound is at least 1.125 greater than the modulating effect of administering the same composition without the excipient.

81. The method of claim 78, wherein the antisense compound targets a mRNA or pre-mRNA.

82. The method of claim 78, wherein the antisense compound targets a non-coding RNA.


SEQUENCE LISTING (8 sequences)

SEQ ID NO: 1 - DNA, 20 bp, Artificial Sequence
  Synthetic Oligonucleotide
ctgctagcctctggatttga

SEQ ID NO: 2 - DNA, 20 bp, Artificial Sequence
  Synthetic Oligonucleotide
tccgtcatcgctcctcaggg

SEQ ID NO: 3 - DNA, 24 bp, Artificial Sequence
  Primer
atgacaatcatgttgcagcaattc

SEQ ID NO: 4 - DNA, 25 bp, Artificial Sequence
  Primer
cgatgcaataaatatgcacaaatca

SEQ ID NO: 5 - DNA, 28 bp, Artificial sequence
  Probe
ctgtaaagctggaaagggacggactggt

SEQ ID NO: 6 - DNA, 15 bp, Artificial sequence
  Primer
tcgccgcttgctgca

SEQ ID NO: 7 - DNA, 17 bp, Artificial sequence
  Primer
atcggccgtgatgtcga

SEQ ID NO: 8 - DNA, 23 bp, Artificial sequence
  Probe
ccatggtcaaccccaccgtgttc